(19) United States
(12) Patent Application Publication
(10) Pub. No.: US 2022/0280067 A1
(43) Pub. Date: Sep. 8, 2022

(54) MEASUREMENT DEVICE THROUGH WHICH BREATHING GAS CAN FLOW FOR MEASURING GAS COMPONENTS OF THE BREATHING GAS

(71) Applicant: HAMILTON MEDICAL AG, Bonaduz (CH)

(72) Inventors: Jürg Roffler, Chur (CH); Felix Breitruck, Buchs (CH)

(73) Assignee: HAMILTON MEDICAL AG, Bonaduz (CH)

(21) Appl. No.: 17/295,487

(22) PCT Filed: Nov. 20, 2019
(86) PCT No.: PCT/EP2019/081992

§ 371 Date: May 20, 2021

(30) Foreign Application Priority Data
Nov. 26, 2018 (DE) 10 2018 129 838.2

(51) Int. Cl. A61B 5/097; G01N 21/05; G01N 21/64; G01N 33/497

(52) U.S. Cl. CPC A61B 5/097; G01N 21/05; G01N 21/645; G01N 33/497; G01N 2021/6432; G01N 2021/6439; G01N 2021/6471; G01N 2021/7786

(57) ABSTRACT

The invention relates to a measurement device for determining at least one gas component of a gas present in a measuring chamber of the measuring device, the measuring device comprising a housing enclosing the measuring chamber, at least one housing wall section of which housing being designed as an observation section for detecting electromagnetic radiation emanating from the observation section in a direction away from the measuring chamber, the observation section comprising at least one film layer, and the housing being designed as a plastic injection-moulded housing. The invention is characterised in that the observation section has at least one observation wall component comprising an injection-moulded observation body injection-moulded onto the at least one film layer, and the housing comprises the at least one observation wall component and an injection-moulded frame body injection-moulded onto the observation wall component.

The present invention concerns a measuring device for the measurement of at least one gas constituent of a gas present in a measuring chamber of the measuring device, where the measuring device comprises a housing that surrounds the measuring chamber, of which at least one housing wall section is configured as an observation section for capturing electromagnetic radiation coming out of the observation section in the direction away from the measuring chamber, where the observation section comprises at least one foil layer and where the housing is configured as a synthetic injection molding housing.

The present invention further concerns a method for fabricating an observation section of such a measuring device, where the observation section encloses the measuring chamber of such a measuring device at least in part.

Measuring devices of the type affected by the present application are also referred to in the state of the art and in the relevant specialist field as “measuring cuvettes” or “measuring gas cuvettes”. They serve in particular in ventilation devices for measuring gas constituents, such as for example CO₂ and/or O₂, in an inhalation gas supplied to a patient to be ventilated or in an exhalation gas produced by same, on the basis of electromagnetic radiation which is characteristic of the particular gas constituent to be investigated and/or that manifests a radiation behavior characteristic of the particular gas constituent to be investigated.

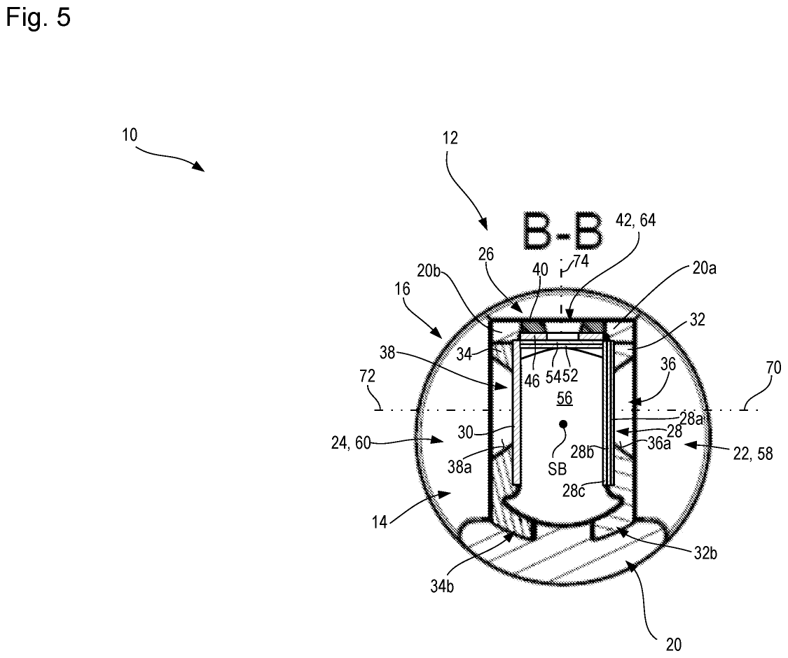

The present application arises in the preamble of of Claim 1 from the embodiment shown in FIG. 5 of U.S. Pat. No. 6,095,986 A. The bidirectional flowthrough measuring device disclosed there shows a synthetic basic housing produced by injection molding. The observation sections through which in the example depicted there infrared radiation for measuring the CO₂ content of gas in the measuring device is radiated into the measuring chamber on one side and exits from the measuring chamber on the other side after passing through the gas-filled measuring chamber, are in the first instance cut out from a basic housing.

Window components are injection-molded from the same synthetic material as the injection-molded basic housing, which after their fabrication are hot-sealed onto an infrared radiation-transparent foil with anti-fog coating. The adequately infrared radiation-transparent foil with anti-fog coating covers a pass-through aperture of the injection-molded window component that passes through same, such that the window component is transparent to infrared radiation, but is impermeable to gas exchange between the measuring chamber and the external environment.

The window components thus bonded with the foil are subsequently inserted in the aforementioned cutouts in the basic housing and bonded to it gas-tight by gluing or ultrasound welding.

This known measuring device has the disadvantage that its production requires a large number of processing steps, where in particular the firm bonding of the window components with the basic housing has to be implemented very accurately in order to indeed produce the desired gas-tightness. Likewise, the process control when hot-sealing the window components to the foils has to be very accurately adjusted and monitored, since between the window components and the foil onto which they are sealed there exist considerable thickness differences, which hamper the fabrication of thermal fused bonding.

A further measuring device for measuring a gas constituent in a gas is known from GB 2 533 806 B. This publication, which deals in particular with avoiding undesirable leaks at the measuring device, points out the leakage problems that arise precisely at the jointing points of thin- and thick-walled sections. In this context, GB 2 533 806 B points out explicitly the problem zone of the window-like observation sections with thin-walled “window pane” and compared with it thick-walled “window frame” for transilluminating the measuring chamber with infrared rays.

GB 2 533 806 B rejects furthermore multistage injection molding methods with the comment that these would not improve the leakage problem of such measuring devices, since the effect of multistage injection molding methods is that component sections fabricated by injection molding at different times solidify at different times, which in turn can cause leakage problems at the bonding points of the component sections fabricated by injection molding at different times.

GB 2 533 806 B therefore proposes fabricating the measuring device in a single injection molding shot and in the thus injection-molded component post-forming observation sections for transilluminating the measuring chamber with infrared radiation through moveable sliders.

Therefore, moveable sliders are driven into the injection-molding cavity that has just been filled with injection molding material, until at the slider's location wall thickness is reached that corresponds to the desired wall thickness of the observation window.

The drawback of the solution proposed by GB 2 533 806 B, however, is that it leaves unsolved the problem of the uptake of the injection molding material which has to be displaced by the sliders driven into the cavity. In technical injection molding terms, the cavity has to be filled completely with injection molding material in order to be able to produce a component with an adequately defined shape with reproducible component quality. If, however, sliders are then driven into the filled cavity in order to displace material in the observation sections, this material has to be able to leave the injection-molding cavity at another point. This results in injection molding tools with complex side-cavities, which have to be opened in a controlled fashion and again closed and emptied.

Injection molding fabrication of the measuring device in a single injection molding shot with sliders already projecting into the cavity is technically not possible without further ado, since according to the data in GB 2 533 806 B the wall thickness of the observation window should not exceed 0.2 mm and preferentially even 0.05 mm. Due to the extension area of the observation window with this small wall thickness, the cavity gap that defines the wall thickness cannot be filled up reliably in injection molding.

A further problem with GB 2 533 806 B lies in the infrared-optical quality of the observation window produced by means of sliders after the injection molding: If the sliders are not exactly matched thermally with the process conditions during injection molding, they extract heat locally from the injection molding material they are in touch with too quickly or too slowly, such that due to thermal distortion, the window area produced by the sliders can deviate from the desired flat shape at least in sections and thus act at least in sections as an entry and/or exit lens. Consequently infrared radiation, which is radiated into the measuring chamber on one side through a window thus formed and on the other side exits again through an opposite window, can in some circumstances undergo multiple refraction, deflection, and/or local focusing, which can impair the quality of the infrared radiation signal received by sensors outside the measuring chamber at the observation section.

A further measuring device is known from DE 10 2006 052 999 A1. This measuring device too, referred to as a “measuring gas cuvette”, serves like the aforementioned measuring devices for capturing the CO₂ fraction in the respiratory gas through infrared spectroscopy or capnometry which are known per se.

The measuring device known from DE 10 2006 052 999 A1 comprises two separately fabricated injection-molded bodies. In their fabrication, a first thin-walled inner injection-molded body is extrusion-coated with an outer thicker-walled further injection-molded body. On opposite sides of the inner injection-molded body, regions are occupied by sliders in which regions injection-molding material of the second outer body should not be able to reach the first body. Thus in the second injection-molding step, observation windows are cut out by sliders at the outer injection-molded body. The cut out windows are covered by the material of the extrusion-coated inner injection-molded body and consequently sealed. With the method known from DE 10 206 052 999 A1, it should be possible to realize wall thicknesses in the range from 170 to 210 μm at the inner injection-molded body that forms the window areas of the observation windows.

The extremely difficult injection-molding fabrication of the thin-walled inner injection-molded body is a drawback of this known measuring device and its fabrication method. In terms of the device, it requires a thermally exactly balanced injection molding form as regards heat capacity and heat conduction, and in terms of the process it requires a considerable cost until the relevant injection molding form is in such thermal equilibrium that the injection-molded body, thin-walled as stated and having a large area relative to its wall thickness, can be manufactured repeatedly with some reliability.

In view of the state of the art expounded above, it is the task of the present invention to propose a technical solution with which a measuring device of the aforementioned type can be produced reliably at the lowest possible cost, with repeatable accuracy, and without undesirable leaks.

According to a device aspect of the present invention, this task is solved by means of a generic measuring device of the aforementioned type in which the observation section exhibits at least one observation wall component, comprising an observation injection-molded body injected onto the at least one foil layer, and where the housing comprises the at least one observation wall component and a frame injection-molded body injected onto the observation wall component .

Through the use of the at least one foil layer, in the observation section of the measuring device a very thin wall region can be provided reliably and with repeatable accuracy. A foil body, comprising one foil layer or a number of laminated and/or coextruded foil layers, can be made thinner than an injection-molded body. Moreover, in particular a number of foil layers can form a ply or foil body configured as multilayer with body properties adjustable within limits in dependence on the utilized foil layers.

Through the injection of the observation injection-molded body onto the at least one foil layer, reliable firm bonding of the observation injection-molded body with the at least one foil layer is already produced at the filling of the injection-molding cavity for the fabrication of the observation injection-molded body, i.e. when the material of the later observation injection-molded body is still flowable and warm or hot. A hot-sealing step as in U.S. Pat. No. 6,095,986 A for the bonding of foil layer and observation injection-molded body can consequently be omitted. Furthermore, in contrast to the known sealing, no external heat source is needed in order to produce sufficiently tight bonding of the observation injection-molded body with the at least one foil layer. Through the injection pressure when injecting the material for forming the observation injection-molded body, moreover, in addition to the necessary amount of heat for forming a gas-tight firm bonding of the observation injection-molded body with the at least one foil layer, the force necessary for forming such bonding is also provided.

The observation wall component thus formed by injecting the observation injection-molded body onto the at least one foil layer can then be completed to the housing of the measuring device in a further injection molding process with the frame injection-molded body. To this end, the at least one already fabricated observation injection-molded body including the foil layer bonded with it should be placed in the cavity for the fabrication of the frame injection-molded body, such that the observation wall component, in particular the observation injection-molded body, can be bathed by the flowable material of the frame injection-molded body and due to the thermal energy plus the high pressure of the frame injection molding material during injection, bonded with the frame injection-molded body.

Thus with the fabrication of the frame injection-molded body, firm bonding between the at least one observation wall component, in particular observation injection-molded body, and the frame injection-molded body can be produced that is completely gas-tight.

Since a thin-walled region of the observation section is already provided with the at least one foil layer, the observation injection-molded body can be implemented nearly arbitrarily thick, such that an adequate wetting area can be provided between the frame injection-molded body injected onto the observation wall component, in particular onto the observation injection-molded body, and the observation wall component for producing firm bonding.

An area of the observation injection-molded body bathed by material of the frame injection-molded body during injection of the frame injection-molded body onto the observation injection-molded body, can deviate from a flat shape in order to increase the size of the area of the observation injection-molded body bathed by material of the frame injection-molded body without having to increase its wall thickness. For example, the bathed area can exhibit a groove, preferentially running along its longitudinal extension or a projection, preferentially running along its longitudinal extension. Then in addition to firm bonding, positive interlocking between the frame injection-molded body and the observation injection-molded body can also be produced, which additionally increases the bonding strength between the aforementioned bodies.

In accordance with the above, the aforementioned task is likewise solved by means of a method for fabricating an observation section, serving the purpose described above and configured for fulfilling this purpose, of a measuring device for measuring a gas constituent of a gas present in a measuring chamber of the measuring device, where the measuring chamber is surrounded at least in part by the observation section, where the method comprises the following steps:
Inlaying of a foil body with at least oner foil layer in an injection-molding cavity,
Injection of an observation injection-molded body onto the foil body and thereby forming an observation wall component,
Injection of a second injection-molded synthetic body onto the observation wall component and thereby fabricating the observation section.

The method serves for the fabrication of at least one observation section, i.e. a section of a housing of a measuring device described above, which a measuring chamber surrounds at least in part. The observation section is suitable for the observation and capture of electromagnetic rays emitted from it to the measuring device.

Of course, it should not be ruled out that with the method described above the entire housing of a measuring device and consequently the entire surrounding of a measuring chamber is produced. This is even preferential. According to the invention, however, for realizing the advantages of the present invention it is already sufficient if by means of the method that section of the measuring device housing is fabricated which makes possible the observation and capture of electromagnetic rays emitted from the observation section of the measuring device. How this observation section is completed into a measuring device housing, is then immaterial. Here it is preferential if the second injection-molded synthetic body is the aforementioned frame injection-molded body and consequently a functional measuring device can be formed with two injection molding processes.

In principle, in the multicomponent injection molding method, in a single injection molding forming tool both the observation injection-molded body can be injected onto the at least one foil layer and also subsequently the second injection-molded synthetic body can be injected onto the observation injection-molded body or onto the observation wall component. Regions of the injection-molding cavity that in the current injection molding step should not just now be reached by injection molding material, can be occupied by sliders.

One obtains greater constructional freedom, however, if the foil body is inlayed in a first injection-molding cavity, in which the observation injection-molded body is injected onto the foil body and thus the observation wall component is formed, and if subsequently the observation wall component is arranged in a second injection-molding cavity different from the first one, in which then the second injection-molded synthetic body is injected onto the at least one observation wall component.

Hereinafter the device and the method are elucidated and further developed together. Device aspects arising from method descriptions are also further developments of the device, and method aspects arising from device descriptions are also further developments of the method.

Boundary areas of the observation wall components form in the, preferentially second, injection-molding cavity for the fabrication of the second injection-molded synthetic body boundary areas of this injection-molding cavity.

A sufficiently robust foil layer that is transparent to electromagnetic radiation in the relevant wavelength range is preferentially made from biaxially oriented polymer. Biaxially oriented polyolefin is especially preferred. In the group of polyolefins, biaxially oriented polypropylene (BOPP) is preferred due to its radiation-transparency and its high mechanical strength even at low wall thicknesses. Preferentially, therefore, the at least one foil layer onto which the observation injection-molded body is injected comprises BOPP. This does not preclude that the foil body on the side of the BOPP foil layer facing away from the observation injection-molded body and/or on the side facing towards the observation injection-molded body exhibit further foil layers or plies, such as e.g. varnish plies, for example in order to decrease the fogging tendency of the foil body in contact with moist gas.

If the foil body exhibits more than one foil layer, it can preferentially exhibit a protective foil layer, in particular made from polycarbonate. Preferentially, the polycarbonate foil layer is arranged on the side facing towards the observation injection-molded body of a polyolefin foil layer, such as e.g. of the aforementioned especially preferential BOPP foil layer. The observation injection-molded body can then be directly injected onto the protective foil layer, in particular onto a protective foil layer made from polycarbonate. In combination with the BOPP foil layer, the protective foil layer can increase the form stability of the foil combination, which is an advantage precisely under the thermal stresses prevailing during injection and the mechanical stresses due to shrinkage during cooling.

Preferentially, the protective foil layer, which does not absolutely have to be made from polycarbonate, but preferentially is the aforementioned polycarbonate foil layer, is thicker than the polyolefin, in particular BOPP foil layer. Preferentially, the protective foil fabricated from polycarbonate is at least 4 to 7 times thicker than the

BOPP foil layer, especially preferentially 5.5 to 6.5 times thicker than the BOPP foil layer.

The BOPP foil layer can exhibit a thickness in the range from 30 to 70 μm, preferentially in the range from 35 to 50 μm. In tests, a BOPP foil layer with a thickness in the range from 40 to 45 μm has proved itself, where according to the latest state of knowledge a thickness of 41 μm is especially preferential.

The protective foil, in particular made from polycarbonate, can exhibit a thickness in the range from 100 to 300 μm, where a thickness range from 230 to 270 pm is preferential. In tests, a polycarbonate foil layer with a thickness in the range from 245 to 255 μm has proved itself, where according to the current state of knowledge a layer thickness of 250 μm is preferred for the protective foil.

For durable bonding of the polyolefin foil layer, in particular BOPP foil layer, with the protective foil layer, in particular polycarbonate foil layer, an adhesion-mediating layer can be arranged between the two foil layers. Preferentially, acrylate adhesive, in particular pure acrylate adhesive, can be arranged between the BOPP foil layer and the polycarbonate foil layer as an adhesion-mediating layer. Such a layer can be arranged as an adhesion-mediating layer between the BOPP foil layer and the protective foil layer as an acrylate adhesive foil or a pure acrylate adhesive foil, for instance with a thickness from 60 to 120 μm, especially preferentially with a thickness of 100 μm.

The observation injection-molded body is preferentially formed from a synthetic based on acrylonitrile butadiene styrene (ABS). Especially preferentially, the synthetic material used to produce the observation injection-molded body is transparent ABS, i.e. for example methyl methacrylate acrylonitrile butadiene styrene (MABS). During the injection molding process it bonds excellently with the aforementioned BOPP foil layer or with the aforementioned polycarbonate foil layer and moreover permits visual inspection of the measuring chamber.

For the fabrication of an ideally gas-tight measuring device with the best possible bonding between an observation wall component, in particular observation injection-molded body, and a frame injection-molded body, the frame injection-molded body is preferentially produced from the same material as the observation injection-molded body.

In principle, any gas present in the measuring chamber can be investigated with the currently discussed measuring device with regard to certain gas constituents. In order to use the measuring device for the analysis of flowing gases, preferentially the measuring chamber allows the flowthrough of gas along a virtual flow path, especially preferentially bidirectionally, such that both inhalation and exhalation respiratory gas can be analyzed. The preferential application of the present measuring device consists in arrangement in a respiratory gas line of a ventilation device and consequently in the analysis of inhalation and exhalation respiratory gas of an artificially ventilated patient.

A possible analysis of the electromagnetic radiation coming out of the observation section serves for the measurement of the CO₂ (carbon dioxide) contained in the observed gas. To this end, use is made in a way that is known per se of infrared radiation that is radiated by an infrared radiation source in such a manner into the measuring chamber that it can be captured in the region of the at least one observation section outside the measuring device's housing. Advantageously, in an observation section configured for CO₂ measurement in the observed gas the at least one foil layer is the only solid body that has to be penetrated by the infrared radiation or generally by the electromagnetic radiation being used. Therefore, it is advantageous for the possible configuration of the measuring device that the observation injection-molded body exhibit an observation cutout inside which the at least one foil layer is accessible. The observation cutout therefore preferentially passes through the observation injection-molded body and is covered by the at least one foil layer. The at least one foil layer forms in this case a window area, as it is known from the state of the art, the observation injection-molded body forming a “window frame”.

In order to be able to fit the observation wall component as precisely as possible into the injection-molding cavity, in which the second synthetic injection-molded body, in particular the frame injection-molded body, is produced by simultaneous injection onto the observation wall component, it can be provided that a lateral face of the observation cutout tapers towards the at least one foil layer and thus is configured as a centering surface for the engagement of a tool, in particular centering tool. The tool engagement can originate from a component of the forming tool which defines the injection-molding cavity of the frame injection-molded body, for instance from a mold core or a slider. The observation cutout can exhibit a polyhedric or generally non-rotation-symmetrical lateral face, such that with the centering a rotational alignment about a virtual cutout axis passing through the observation cutout is also possible. In principle, however, a conically tapering observation cutout is also possible. For measuring the CO₂ fraction in the gas that is to be observed in the measuring chamber, the at least one foil layer is preferentially transparent to electromagnetic radiation in the infrared spectral range.

In principle it is possible that a radiation source, for example an infrared radiation source, is arranged in the measuring chamber and from there trans-irradiates the gas present in the measuring chamber and also the observation section located behind the gas as seen from the radiation source. In this case it suffices if the measuring device exhibits a single observation section or a single observation injection-molded body with at least oner foil layer.

It is precisely in the measurement of the CO₂ content of the gas in the measuring chamber, however, that it is advantageous if the gas in the measuring chamber is as undisturbed as possible by a radiation source inside the measuring chamber that during operation also produces heat. Preferentially, therefore, the measuring chamber is trans-irradiated with infrared radiation or generally with electromagnetic radiation. In order to configure the measuring chamber such that it can be trans-irradiated with electromagnetic radiation, it can be provided that the measuring device comprises a first and a second observation wall component, which are provided for the capture of a first gas constituent in such a way at the housing that at least one section of the measuring chamber is located between them. In order to distinguish these observation wall components from other observation wall components that are configured and/or provided for capturing some other second gas constituent, the currently discussed observation wall components are referred to as “first gas constituent observation wall components”.

The first and the second first gas constituent observation wall components are arranged at the housing in such a way that electromagnetic radiation, in particular light, especially preferentially infrared radiation, which radiates into the measuring chamber through an observation section comprising one of the two first gas constituent observation wall components, and can radiate back out of the measuring chamber through the observation section of the respectively other first gas constituent observation wall component.

In the measurement of gas constituents of a gas in the measuring chamber, the temperature of the gas to be observed can play an important part.

The measuring device can additionally or alternatively to the aforementioned observation section, which is configured for trans-irradiation of the gas in the measuring chamber with electromagnetic radiation, exhibit an observation section for measuring the gas temperature. To this end, the at least one foil layer of a thus formed temperature observation section can comprise a metal foil as a temperature measurement foil layer. The metal foil, which usually exhibits a thickness below 20 μm, preferentially below 10 μm, is on the one hand gas-tight, and on the other exhibits, compared with synthetic foils, high thermal conductivity. Preferentially, the metal foil is an aluminum foil that is not susceptible to oxidation, is easily available, and is a very good heat conductor. The temperature measurement foil layer and the observation injection-molded body injected onto it form a temperature observation wall component.

For improved capture of the thermal radiation emitted by the metal foil of the temperature measurement foil layer, it can be coated in the observation section on its side that faces away from the measuring chamber with black material, for instance black varnish or black synthetic material. The thus formed temperature measurement foil layer acts when viewed from outside the measuring chamber almost like an ideal black body, such that due to the low thickness of the metal foil and its high thermal conductivity, by capturing the thermal radiation emitted from the black coating it is possible to deduce very accurately the temperature of the gas in the measuring chamber.

The temperature measurement foil layer can, like the foil layer made preferentially from BOPP described above, be accessible from outside the measuring chamber, i.e. from outside measuring device, through an observation cutout. The above also applies here: The observation cutout can pass through the observation injection-molded body in the thickness direction and be covered by the temperature measurement foil layer. For accurate arrangement of the temperature observation wall components in a further injection-molding cavity, the border of the observation cutout can be configured, as described above, to taper towards the temperature measurement foil layer.

The temperature observation wall component described above can also, additionally or alternatively to the synthetic foil preferentially made of BOPP, exhibit the described temperature measurement foil layer. If the temperature observation wall component exhibiting the temperature measurement foil layer also exhibits a synthetic foil, in particular made of BOPP, it is arranged preferentially between the observation injection-molded body and the temperature measurement foil layer. To facilitate the injection of the observation injection-molded body onto the preferentially metallic temperature measurement foil layer, the temperature measurement foil layer can exhibit a primer layer or before the injection molding be laminated with the synthetic foil into a composite body. The observation injection-molded body is then injected onto the synthetic foil layer of the composite body. To minimize as far as possible the effect of the thermal radiation emitted from the temperature measurement foil layer, the synthetic foil is preferentially cut away in the region of the observation cutout.

Although the state of the art mentioned at the outset relates solely to the measurement of the CO₂ content in a gas, it is by no means the case that CO₂ is the only gas constituent that can be captured through observation and/or capture of electromagnetic radiation emitted from an observation section. Additionally or alternatively, for capturing a second gas constituent different from the first one it can be provided that the at least one foil layer exhibit as a second gas constituent foil layer for capturing the second gas constituent a photoluminescent layer with at least one luminophore accommodated in it.

Certain gas constituents, such as for example O₂ (oxygen), have the property of quenching the radiation of a luminophore bathed by the gas and excited by an external radiation source to emit radiation as a function of its concentration in the gas. Through this quenching, there is a change in the duration and/or the intensity of the radiation of the luminophore in the photoluminescent layer excited by the external radiation source to emit radiation as a function of the concentration of the quenching gas constituent coming into contact with the luminophore.

The denotation first gas constituent and second gas constituent serve here solely for distinguishing between the gas constituents. The measuring device could exhibit just the second gas constituent foil layer and consequently be configured only for measuring the second gas constituent by means of photoluminescence.

Since it is precisely in photoluminescence-based methods for measuring a gas constituent of a gas being observed, that its temperature plays an important part, the measuring device configured for measuring the second gas constituent preferentially exhibits a second gas constituent observation wall component with the second gas constituent foil layer, which also exhibits the temperature measurement foil layer.

In order to prevent the radiation of the photoluminescent layer being affected by a gas constituent of the atmosphere surrounding the measuring device outside the measuring chamber, the second gas constituent foil layer can be covered completely on its side facing away from the measuring chamber by the observation injection-molded body. To render the signal of the photoluminescent layer analyzable, the observation injection-molded body of the second gas constituent observation wall component is transparent to electromagnetic radiation in the wavelength range of the photoluminescent radiation emitted by the photoluminescent layer. Since the photoluminescent layer radiates not only electromagnetic radiation in a predefined wavelength range, but also has to be excited to radiate through electromagnetic radiation in an excitation wavelength range differing from the radiated wavelength range, the observation injection-molded body is preferentially transparent also to electromagnetic radiation in the wavelength range of the excitation radiation exciting the photoluminescent layer, in order to make possible this excitation from outside the measuring chamber.

Due to the fact that the observation injection-molded body can be configured with an almost arbitrary wall thickness, it can be ensured that a second gas constituent present in the atmosphere surrounding the measuring device, for example oxygen, does not penetrate the observation injection-molded body up to the photoluminescent layer, such that the quenching to be observed of the radiated photoluminescent radiation is based solely on the second gas constituent of the gas in the measuring chamber.

Preferentially, the measuring device is configured for measuring both the first gas constituent and the second gas constituent. To this end, the measuring device can exhibit both the first and the second first gas constituent observation wall component and also a second gas constituent observation wall component with the second gas constituent foil layer.

In order to create a spatially compact measuring device, which nevertheless can measure two different gas constituents in the gas in the measuring chamber, the first and the second first gas constituent observation wall component are each arranged at the housing on a different side of the second gas constituent observation wall component. If, as is preferential, the measuring chamber allows the flowthrough of gas along the aforementioned flow path, preferentially the two first gas constituent observation wall components and the second gas constituent observation wall component overlap along the flow path at least in part, preferentially completely.

For use in a gas line system, preferentially a connector formation is arranged at least at one longitudinal end of the housing, configured for connecting a hose and/or pipe line. Since the housing is fabricated by injection molding, preferentially the connector formation is configured integrally with an observation region of the measuring device that surrounds the measuring chamber. The observation region comprises a number of observation sections, preferentially all observation sections.

Preferentially, one such a connector formation is arranged at each of the two longitudinal ends of the housing. The connector formation is configured in particular for connecting a respiratory gas line of a ventilation device.

Since the present invention with the observation injection-molded body, which is injected onto at least one foil layer, can provide an especially versatile wall component as a basic building block for fabricating an aforementioned measuring device, with which the measuring device can be configured for measuring different gas constituents, the present application also concerns an observation wall component designed as described above for a measuring device configured as described above. Such an observation wall component exhibits a foil layer with an observation injection-molded body injected onto it, where the foil layer exhibits a photoluminescent layer with at least one luminophore accommodated on it and/or where the observation injection-molded body exhibits an observation cutout inside which the at least one foil layer is accessible, where a lateral face of the observation cutout tapers in the direction towards at least one foil layer and thus is configured as a centering surface for the engagement of a tool.

The present invention concerns in general also a measuring device for measuring at least one gas constituent of a gas present in a measuring chamber of the measuring device, where the measuring device comprises a housing surrounding the measuring chamber, of which at least one housing wall section is configured as an observation section for capturing electromagnetic radiation emitted from the observation section in a direction away from the measuring chamber, where the observation section comprises at least one window component which at least in part, preferentially completely, is made of glass transparent to the electromagnetic radiation and where the housing is configured as a synthetic injection-molded housing.

Such a measuring device can be fabricable reliably at the lowest possible cost, with repeatable accuracy, and without undesirable leaks as a consequence of the observation section exhibiting at least one observation wall component, where the observation wall component comprises an observation injection-molded body, of which at least one section of the window component is extrusion-coated, where preferentially the housing comprises the at least one observation wall component and a frame injection-molded body injected onto the observation wall component.

Due to the transparency to infrared radiation, the window component can be used to measure the CO₂ fraction of the gas if the glass of the window component is made from aluminum oxide or germanium. The window component can therefore comprise sapphire glass or chalcogenide glass.

For an equally gas-tight as it is mechanically firm bonding of the window component with the observation injection-molded body, the window component can preferentially be extrusion-coated by the material of the observation injection-molded body along its entire outer circumference. For the purpose of achieving an equally firm as it is tight arrangement of the window component in an observation cutout of the observation injection-molded body, the window component can exhibit at its outer circumference at least along a circumferential section, preferentially along its entire outer circumference, an anchor projection protruding from a radially further inwards located window section towards radially outwards.

Preferentially the anchor projection is configured as thinner in the thickness direction than the window section from which it protrudes radially outwards. This makes possible the preferential arrangement of the window component extrusion-coated by material of the observation injection-molded body in such a way that the internal side of the window component or of the window section facing towards the measuring chamber is arranged flush with the inner surface of the observation injection-molded body surrounding the window component. Thus on the one hand, material of the observation injection-molded body can be present on both sides of the anchor projection in the thickness direction, which anchors the window component firmly in the observation injection-molded body. On the other, an observation section with an essentially smooth, projection-free inner surface can be obtained, such that an undesirable accumulation of moisture in the transition region between the observation injection-molded body and the window component can be avoided.

Especially advantageously, the anchor projection is configured as flush with the outer surface of the window section and at the outer surface joins the window section without steps. A step between the anchor projection and the window section is then present solely on the internal side of the window component, where preferentially the anchor projection is set back in the thickness direction relative to the inner surface of the window section. Hereby, with a stable anchor projection and with a flush arrangement of the inner surface of the window section and the inner surface of the observation injection-molded body, a maximum quantity of material of the observation injection-molded body can be arranged between the anchor projection and the inner surface of the observation injection-molded body or of the measuring chamber. This increases the firmness of the bonding between the observation injection-molded body and the window component.

Preferentially, the internal side and the external side of the window component form front faces of the window component that are parallel to each other. The anchor projection is then configured at the circumferential lateral surface of the window component. Preferentially, the internal side and the external side of the window component are parallel to each other, in order to avoid undesirable optical refraction effects.

On the external side of the observation injection-molded body, the arrangement of sufficient material of the observation injection-molded body between the outer surface of the observation injection-molded body and the window component or the anchor projection is not a problem, since no flush outer surface is needed on the external side of the measuring chamber.

This measuring device too, can be further developed in accordance with one of the Claims 2 to 4, with the proviso that in the claim wording “the at least one foil layer” should be replaced by “the window component”.

The window component can be used together with the observation injection-molded body to form a first and a second first gas constituent observation wall component, as described and further developed above. Again, it is the case that according to a preferential further development of the invention, at the housing the first and the second first gas constituent observation wall component are each arranged on a different side of the second gas constituent observation wall components.

The present invention also concerns an observation wall component for a measuring device designed as described above with a glass-containing window component, where the observation injection-molded body exhibits an observation cutout within which the window component is accessible, where preferentially a lateral face of the observation cutout tapers in the direction towards at least one foil layer and thus is configured as a centering surface for tool engagement.

The anchor projection can also comprise anchor part-projections spaced away from each other in the circumferential direction.

The present invention is described in more detail below with the help of the enclosed drawings. They show:

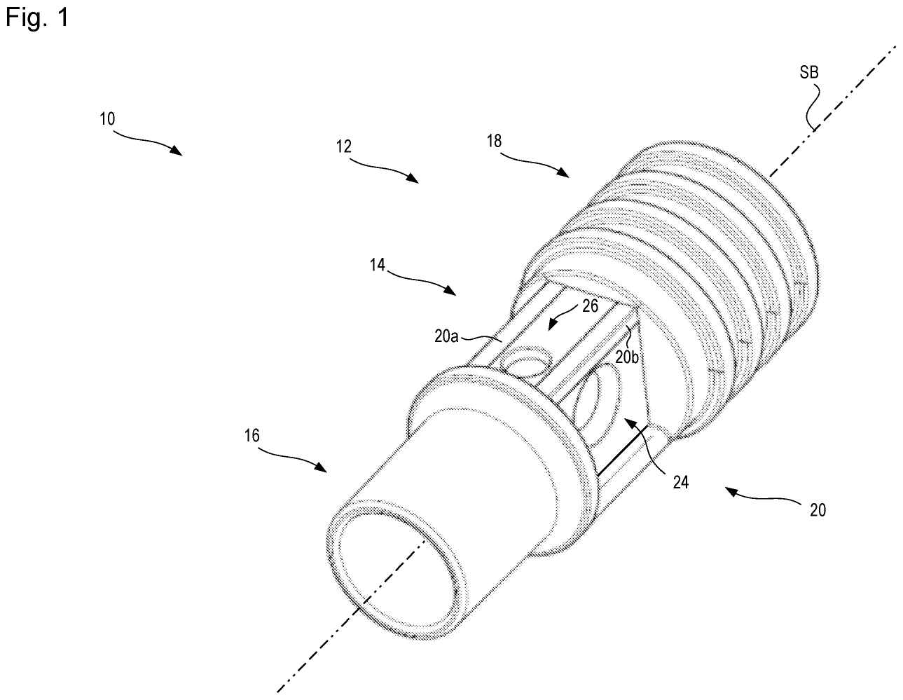

FIG. 1A perspective view of an embodiment according to the invention of a measuring device of the present application,

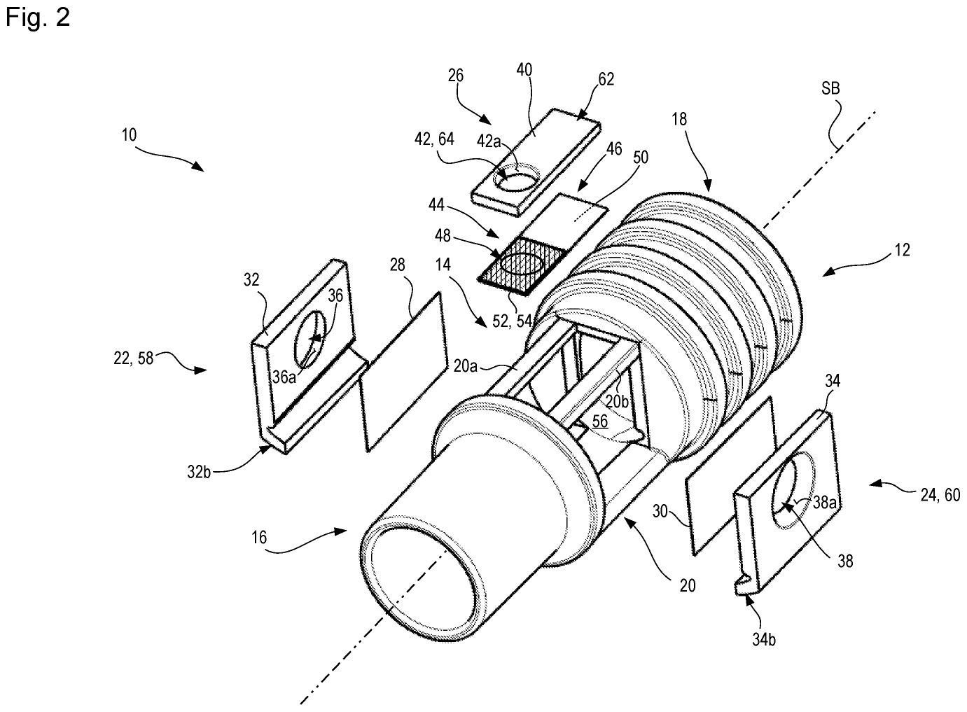

FIG. 2 The measuring device of FIG. 1 in an exploded view in perspective,

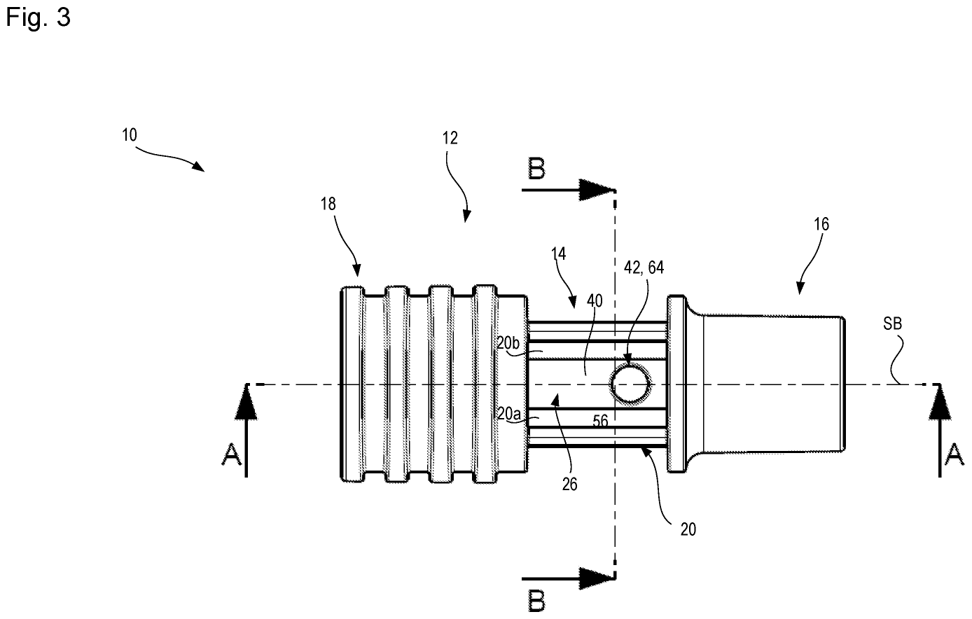

FIG. 3A top view of the measuring device of FIGS. 1 and 2,

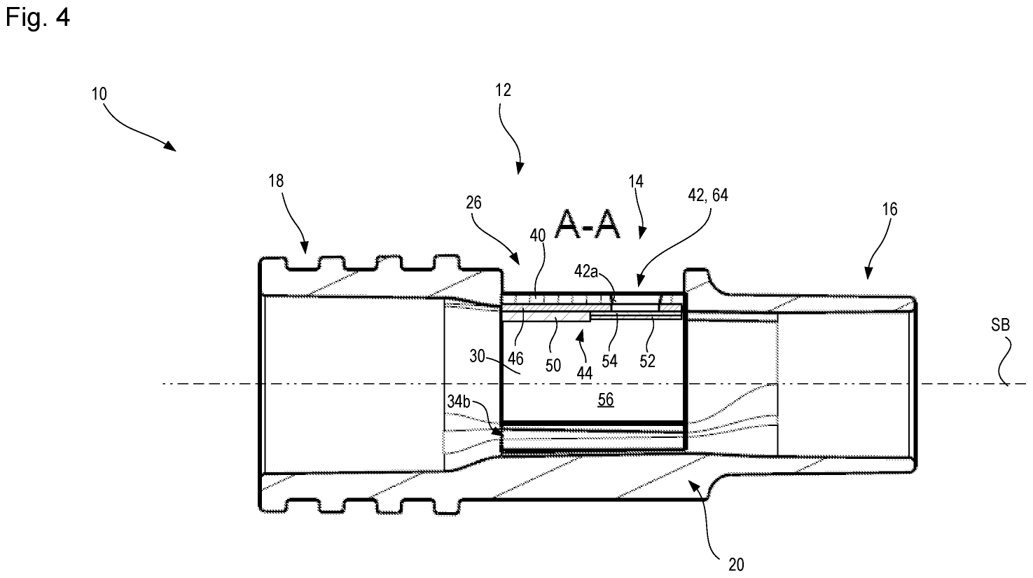

FIG. 4A sectional view through the measuring device of FIGS. 1 to 3 along the section plane A-A of FIG. 3,

FIG. 5A sectional view of the measuring device of FIGS. 1 to 4 along the section plane B-B of FIG. 3,

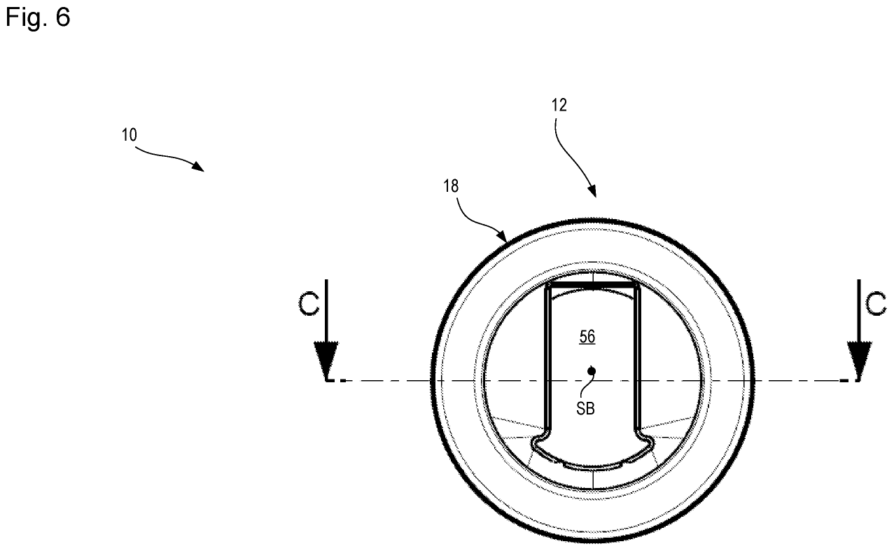

FIG. 6A view of the measuring device of FIGS. 1 to 5 along the flow path in the direction of an expiratory respiratory gas flow,

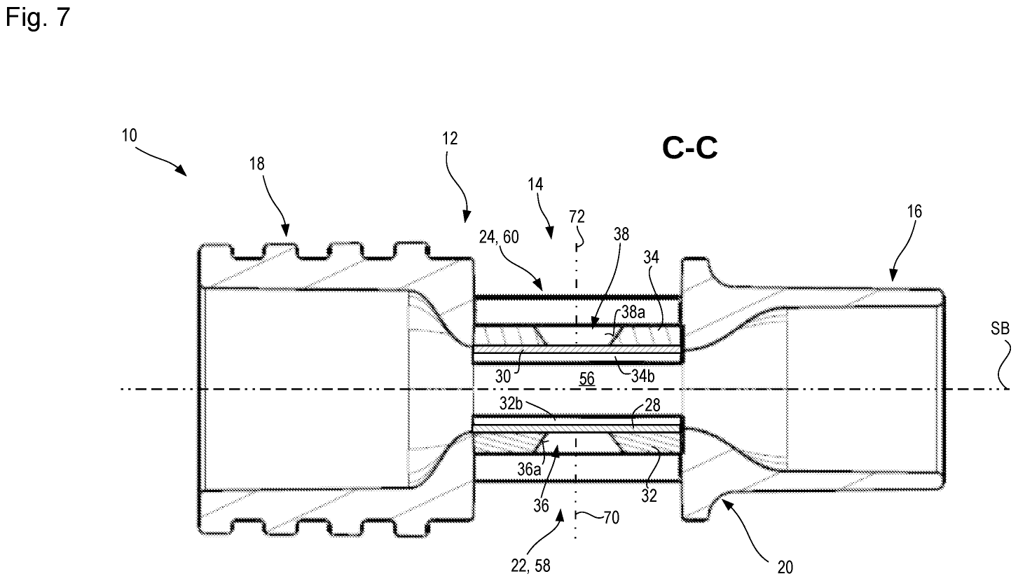

FIG. 7A sectional view through the measuring device of FIGS. 1 to 6 along the section plane C-C of FIG. 6, and

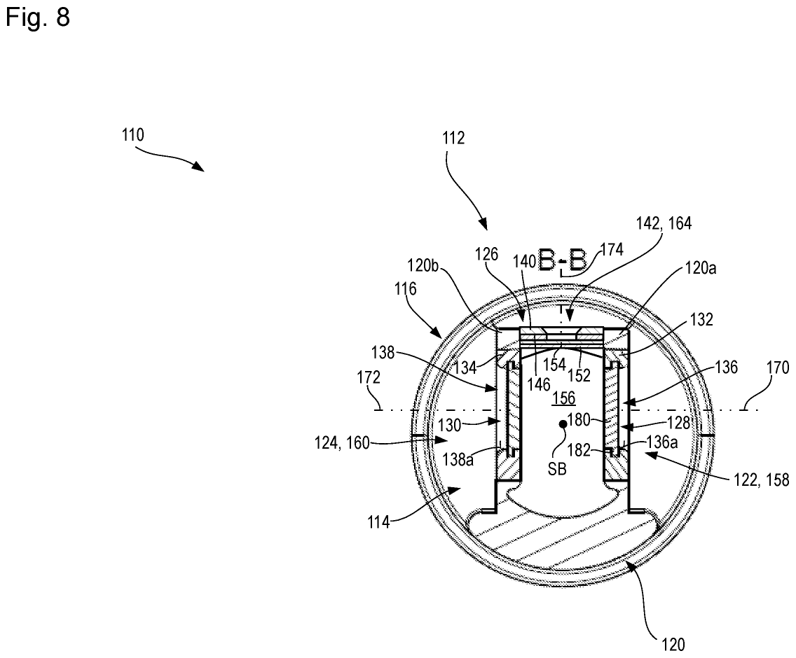

FIG. 8A sectional view of a second embodiment of a measuring device corresponding to the view of FIG. 5.

In FIGS. 1 to 7, an embodiment according to the invention of a measuring device is denoted generally by 10. The measuring device 10 serves to measure at least one gas constituent of a gas flowing through the measuring device 10. The measuring device 10 comprises a synthetic housing 12, through which flow is possible bidirectionally along a virtual flow path SB which in the depicted example is preferentially straight-lined.

The housing 12 comprises a central observation region 14, a distal connector formation 16 for connecting a gas line, in particular a ventilation hose, and a proximal connector formation 18 for connecting a gas-carrying line, in particular a ventilation hose or ventilation tube.

The measuring device 10, configured for use in a ventilation hose arrangement of a ventilation device for artificial ventilation of patients, is normally connected with the distal connector formation 16 with the respiratory gas source of the ventilation device and is connected via the proximal connector formation 18 with the patient to be ventilated.

The housing 12 exhibits a frame injection-molded body 20 and in the present example three observation wall components 22, 24, and 26, of which in FIG. 1 only the observation wall components 24 and 26 can be discerned.

FIG. 2 shows an exploded view in perspective of the measuring device 10 of FIG. 1.

FIG. 2 shows also the observation wall component 22 that is not discernible in FIG. 1. Likewise it is discernible that the frame injection-molded body is a component formed integrally through injection molding, at which the connector formations 16 and 18 are configured.

The two observation wall components 22 and 24 serve at the measuring device 10 for the measurement of CO₂ as a first gas constituent. They are, therefore, first gas constituent observation wall components 22 and 24. They are preferentially configured as mirror-imaged relative to a plane orthogonal to the flow path SB, such that each wall component 22 and 24 can be used as a first gas constituent observation wall component of the measuring device 10 on each side of the frame injection-molded body 20. Each of the two first gas constituent observation wall components 22 and 24 exhibits a foil body 28 or 30 respectively, which is made from BOPP or at least exhibits a BOPP layer on its side that faces away from the flow path SB.

The foil body 28 is shown in detail as an example and not to scale. In the depicted example, the foil body 28 comprises, from the outside inwards, a protective foil 28a preferentially made from polycarbonate, an adhesion-mediating layer 28b, preferentially made from pure acrylate adhesive, and a BOPP foil layer 28c. The foil body 30 can be constructed identically to foil body 28. Differently from the depiction, the foil body 28 can exhibit just one foil layer. Then the foil body 28 can be constructed identically to the foil body 30 shown as an example.

An observation injection-molded body 32 is injected onto the polycarbonate layer 28a of the foil body 28. An observation injection-molded body is injected onto the BOPP layer of the foil body 30. To this end, the foil bodies 28 and 30 are inserted respectively into an injection-molding cavity and then the observation injection-molded bodies 32 and 34 injected onto the foil body 28 or 30 respectively in an injection molding process.

Each of the observation injection-molded bodies 32 and 34 exhibits a cutout 36 or 38 respectively that passes through it in the thickness direction, which is covered at its end that is located nearer to the virtual flow path SB by the respectively assigned foil body 28 or 30 respectively.

A lateral face 36a of the observation cutout 36 and a lateral face 38a of the observation cutout 38 are each configured as tapering, preferentially conically, towards the foil body 28 or 30, such that each lateral face 36a and 38a can respectively serve as a centering surface for the arrangement of the wall component 22 or 24 respectively in an injection-molding cavity for fabrication of the frame injection-molded body 20. The wall component 22 can then be centered by reference to its lateral face 36a and the wall component 24 by reference to its lateral face 38a. Hereby a very accurate arrangement of the wall components 22 and 24 in the injection-molding cavity for fabrication of the frame injection-molded body 20 is possible.

Due to the preferentially mirror-symmetrical configuration to a plane orthogonal to the virtual flow path SB, the cutouts 36 and 38 in the depicted embodiment are located in the longitudinal middle of the observation injection-molded body 32 or 34 respectively relative to the virtual flow path SB.

To facilitate their arrangement at frame injection-molded body 20 and in particular for arrangement in the injection-molding cavity for fabrication of the frame injection-molded body 20, the observation injection-molded body 32 and 34 each exhibit at their respective lower ends as shown in FIGS. 1 and 2 an alignment formation 32b or 34b respectively.

Although FIG. 2 shows an exploded view of the measuring device 10, it should be understood that due to the injection of the frame injection molding body 20 onto the observation wall components 22, 24, and 26 the measuring device 10 thus formed can no longer be disassembled, but rather that frame injection-molded body 20 and the observation wall components 22, 24, and 26 form an essentially firmly bonded unit.

The first gas constituent observation wall components 22 and 24 each exhibit both on their side that in the finished installed state faces towards the virtual flow path SB, at which the foil bodies 28 or 30 respectively are located, and on their external side facing away from the virtual flow path SB, which is formed by an external side of the observation injection molding body 32 or 34 respectively, a plane outer surface each.

The wall component 26 serves in the depicted embodiment for measuring an O₂ fraction in the gas flowing through the measuring device 10. The wall component 26 is therefore in distinction from the first gas constituent observation wall components 22 and 24 a second gas constituent observation wall component 26 in terms of the present application and exhibits an observation injection-molded body 40, which in the thickness direction has a cutout 42 passing through it.

The observation injection-molded body 40, like the other two observation injection-molded bodies 32 and 34, injected by means of injection molding onto a foil body, here the foil body 44.

In contrast to the foil bodies 28 and 30, which can comprise just a single BOPP layer, the foil body 44 is multilayered. The foil bodies 28 and 30, however, can also be configured as multilayered.

The foil layer of the foil body 44 lying next to the observation injection-molded body 40 is a BOPP foil layer 46. The BOPP foil layer 46 exhibits there a cutout 48 passing through it, where after the injection of the observation injection-molded body 40 its cutout 42 is located.

On the side of the BOPP foil 46 facing away from the observation injection-molded body 40 there is located a luminophore-containing foil 50 in a region of the BOPP foil 46 lying nearer to the proximal connector formation 18. The luminophore in the luminophore-containing foil layer 50 can be excited through the preferentially transparent observation injection-molded body 40 to radiate electromagnetic radiation, in particular light, and can be observed through the observation injection-molded body 40 in its excited radiation behavior.

Oxygen in the gas flowing through the measuring device 10 along the virtual flow path SB comes into contact with the excited luminophore of foil layer 50, whereby the luminophore is quenched, i.e. ‘de-excited’, and consequently its radiation behavior changes as a function of the oxygen concentration in the gas flowing through the measuring device 10.

In a section of the foil body 44 lying nearer by the distal connector formation 16, it exhibits on a side of the BOPP foil layer 46 facing away from the observation injection-molded body 40 a metal foil 52, which on its side facing towards the BOPP foil layer 46 and consequently towards the observation injection-molded body 40 is coated with black varnish 54.

The metal foil 52 coated with black varnish 54 consequently forms a temperature measurement foil layer, which is observable through the cutout 42. The metal foil 52, preferentially an aluminum foil 52, with likewise preferentially a material thickness in the single-digit pm range, due to its good heat conductance adopts the temperature of the gas flowing through the measuring device 10 along the virtual flow path SB. The black varnish 54 emits thermal radiation characteristic of the temperature of the metal foil 52 through the cutout 42 towards the outside, where it is observable as infrared radiation. In this way the temperature of the gas flowing through the measuring device 10 can be measured. The wall component 26 is, therefore, also a temperature observation wall component 26.

Through the cutouts 36 and 38, the measuring device 10, again more accurately a measuring chamber 56 in the interior of the measuring device 10 surrounded by the observation wall components 22, 24, and 26, can be penetrated by infrared rays. To this end, infrared radiation is sent with an external infrared radiation source through one of the cutouts 36 or 38 into the measuring chamber 56 in such a way that the infrared radiation is observable through the respective other cutout. The infrared spectroscopic method that can be used to measure the carbon dioxide content of the respiratory gas is sufficiently known.

Since the two observation wall components 22 and 24 are essentially constructed identically, each of the two wall components 22 and 24 can be used to send infrared radiation into the measuring chamber 56 and the respective other to observe the infrared radiation emerging from the measuring chamber 56 after passing through it.

Therefore, the first first gas constituent observation wall component 22 forms an observation section 58 and the second first gas constituent observation wall component 24 an observation section 60.

The observation wall component 26 comprises in contrast two separate observation sections, namely an observation section 62 for observing the radiation behavior of the luminophore of the luminophore-containing foil layer 50 and a temperature observation section 64 in the region of the cutout 42 for observing the thermal radiation emitted from the temperature measurement foil layer 52 with varnish application 54 for determining the temperature of the gas flowing through the measuring device 10. It is also possible for just one of the observation sections 62 and 64 to be provided.

Furthermore, the lateral face 42a of the cutout 42 in the observation injection-molded body 26 is also configured as tapering conically from outside towards the foil arrangement 44, in order to be able to arrange the observation wall component 26 that is usable both for temperature measurement and for measuring the oxygen content positioned accurately and centered in the injection-molding cavity for fabrication of the frame injection-molded body 20.

The observation injection-molded body 40 of the observation wall component 26 is bounded by a plane surface both on its side that faces towards the measuring chamber 56 and also on its external side that faces away from the measuring chamber 56. Preferentially, the two plane boundary areas are parallel to each other. This also applies to the aforementioned observation injection-molded body 32, 34. Due to differing layer thicknesses of the metal foil 52 provided with the black varnish layer 54 on the one hand and the luminophore-containing foil layer 50 on the other, the boundary area of the observation wall component 26 facing towards the measuring chamber 56 can exhibit two, preferentially plane, area sections which are offset relative to each other in a direction orthogonal to the flow path SB.

In the sectional view of FIG. 4, in which the observer looks through the measuring chamber 56 towards the BOPP foil layer 30, the foil body 44 of the observation wall component 26 is discernible in top view.

One can discern in particular that the foil body 44 does not have to exhibit uniform thickness. For example, in the region of the luminophore-containing foil layer 50 it can be configured as thicker than in the region of the temperature measurement foil layer 52 with the black coating 54 on it.

It is discernible in FIG. 5 how many surfaces of the observation wall parts 22, 24, and 26 are wetted by surfaces of the frame injection-molded body 20 during injection molding fabrication of the frame injection-molded body 20, such that firm bonding is produced between the observation injection-molded bodies 32, 34, and 40 that preferentially are produced from at least compatible, preferentially identical synthetics on the one hand and the frame injection-molded body 20 on the other. In particular the alignment formations 32b and 34b of the observation injection-molded bodies 32 or 34 respectively exhibit a large area wetted by the frame injection-molded body 20, which is even angled and encompasses an outer surface of the alignment formations 32b and 34b.

FIG. 5 depicts the virtual cutout axes 70, 72, and 74 of the cutouts 36, 38, and 42 respectively. The cutouts 36, 38, and 42 taper along the virtual cutout axes 70, 72, and 74 respectively that pass through them centrally from outside the measuring chamber 56 in the direction towards the measuring chamber 56. The cutout axes 70, 72, and 74 can be conus axes of the conical lateral faces 36a, 38a, and 42a of the relevant cutouts 36, 38, and 42 respectively, for instance if the respective lateral faces are configured as rotation-symmetrical.

Since the two observation sections 58 and 60 are configured for trans-irradiation of the measuring chamber 56 arranged between them, preferentially the associated cutout axes 70 and 72 are collinear.

As FIG. 5 further shows, the second gas constituent wall component 26 is located between the two first gas constituent observation wall components 22 and 24, or in other words: The first gas constituent observation wall components 22 and 24 are each located on different sides of the second gas constituent observation wall component 26. Preferentially the extension regions of the wall components 22, 24, and 26 overlap along the virtual flow path SB, such that the measuring chamber 56 can be configured to be short along the flow path SB.

In FIG. 5 it is further discernible how the outer surfaces of the observation injection molding components 32, 34, and 40 that face away from the measuring chamber 56 are configured as planar and are arranged parallel or at a right angle to each other. The crosspieces 20a and 20b of the frame injection-molded body 20 in the region of the measuring chamber 56 running parallel to the flow path SB, arranged between each of the first gas constituent observation wall components 22 or 24 respectively on the one hand and the second gas constituent observation wall component 26 on the other, connect flush with their outer surfaces with the outer surfaces of the wall components 22, 24, and 26, such that a measurement device coming from the side of the wall component 26 in the observation region 14 of the measuring device 10 orthogonally to the virtual flow path SB can be pushed onto same. Such a measuring device can exhibit an infrared radiation source and an infrared sensor located opposite to this source in a direction orthogonal to the virtual flow path SB. Furthermore, the measuring device can exhibit between the infrared radiation source and the infrared sensor an excitation radiation source for excitation of the luminophore in the luminophore-containing foil layer 50 and a capture device provided for observing the quenching behavior of same. Likewise, a further infrared sensor for capturing the thermal radiation emitted from the temperature measurement foil layer 52 can be arranged therein. Such a measuring device can simply be pushed onto the observation region 14 of the measuring device 10 and again removed from it.

FIG. 7 shows a sectional view through the measuring device 10 along the section plane C-C of FIG. 6. The two foil bodies 28 and 30 are aligned parallel to the virtual flow path SB and parallel to each other.

FIG. 8 shows a sectional view of a second embodiment of a measuring device 110 in the section plane B-B of FIG. 3, however in the opposite direction of view towards the section plane B-B to the one indicated in FIG. 3. Basically, the view of FIG. 8 corresponds in the section plane to the depiction of FIG. 5. Identical and/or functionally identical components and component sections as in the first embodiment of FIGS. 1 to 7 are provided in FIG. 8 with the same reference labels, but increased numerically by 100.

The second embodiment of FIG. 8 will only be described hereunder in so far as it differs from the first embodiment of FIGS. 1 to 7 whose description otherwise also applies to the second embodiment of FIG. 8.

A rather insignificant difference consists in the fact that the observation injection-molded bodies 132 and 134 do not exhibit alignment formations at their end regions distal to the observation wall component 126.

In the observation cutouts 136 and 138, instead of foil bodies there are present window components 128 or 130 respectively made of glass. The window components 128 and 130 are configured identically and merely arranged in the measuring device 110 mirror-inverted relative to each other by reference to a mirror-symmetry plane spanned by the flow path SB and the cutout axis 174. The window components 128 and 130 exhibit in the depicted example a circular circumference.

This, however, need not be the case. The window components 128 and 130 can alternatively exhibit an oval or a polygonal circumference.

Due to their identical configuration, it suffices to describe the window component 128 in more detail hereunder. Its description also applies to the window component 130, allowing for the aforementioned mirror symmetry.

The window component 128 exhibits a central window section 180, which in the described example exhibits an essentially cylindrical shape, where preferentially the cutout axis 170 is the cylindrical axis of the window section 180.

Running completely around the window section 180, the window component 128 exhibits an anchor projection 182 protruding radially from the window section 180, which exhibits a lower thickness than the window section 180. The anchor projection 182 is surrounded along its entire circuit around the cutout axis 170 on three sides by U-shaped material of the observation injection-molded body 132. The window component 180 with its anchor projection 182 was extrusion-coated by same during the injection molding fabrication of the observation injection-molded body 132. As a result, both mechanically firm and gas-tight bonding of the window component 128 with the observation injection-molded body 132 was achieved.

The window components 128 and 130 are made in the present case of sapphire glass or chalcogenide glass, i.e. they consist either of aluminum oxide or of germanium.

The inner surface of window section 180 of window component 128 is arranged flush with the inner surface of the observation injection-molded body 132. As a result, steps at the inner walls bounding the measuring chamber 156 are avoided, at which otherwise moisture could undesirably condense.

The anchor projection 182 forms together with the window section 180 a plane outer surface of the window component 128.

1. A measuring device for measuring at least one gas constituent of a gas present in a measuring chamber of the measuring device, where the measuring device comprises:
a housing surrounding the measuring chamber, of which at least one housing wall section is configured as an observation section for the acquisition of electromagnetic radiation emitted from the observation section in a direction away from the measuring chamber, where the observation section comprises at least one foil layer and where the housing is configured as a synthetic material injection molded housing,
wherein the observation section exhibits at least one observation wall component, comprising an observation injection-molded body injected onto the at least one foil layer, where the housing comprises the at least one observation wall component and a frame injection-molded body injected onto the observation wall component.

2. The measuring device according to claim 1,
wherein the measuring chamber allows the flowthrough of gas along a virtual flow path.

3. The measuring device according to claim 1,
wherein the observation injection-molded body exhibits an observation cutout, within which the at least one foil layer is accessible.

4. The measuring device according to claim 3,
wherein a lateral face of the observation cutout tapers in a direction towards at least one foil layer and thus is configured as a centering surface for tool engagement.

5. The measuring device according to claim 1,
wherein the at least one foil layer is transparent to electromagnetic radiation in the infrared spectral range.

6. The measuring device according to claim 5,
wherein the at least one foil layer comprises a biaxially oriented polymer foil.

7. The measuring device according claim 1,
wherein it comprises a first and a second first gas constituent observation wall component, which are provided for capturing a first gas constituent at the housing in such a way that at least one section of the measuring chamber is located between them, where the first and the second first gas constituent observation wall component are arranged in such a way at the housing that electromagnetic radiation which is radiated into the measuring chamber through an observation section of one of the two first gas constituent observation wall components, can radiate out of the measuring chamber through the observation section of the respectively other first gas constituent observation wall component.

8. The measuring device according to claim 1,
wherein the at least one foil layer comprises as a temperature measurement foil layer a metal foil.

9. The measuring device according to claim 1,
wherein the at least one foil layer exhibits as second gas constituent foil layer for capturing a second gas constituent differing from the first one a photoluminescent layer with at least one luminophore accommodated in it.

10. The measuring device according to one of the claims 8 and 9,
wherein it exhibits a second gas constituent observation wall component with the second gas constituent foil layer, which also exhibits the temperature measurement foil layer.

11. The measuring device according to one of the claim 9 or 10,
wherein the second gas constituent foil layer is covered completely by the observation injection-molded body on its side facing away from the measuring chamber, where the observation injection-molded body is transparent to electromagnetic radiation in the wavelength range of the photoluminescent radiation emitted from the photoluminescent layer.

12. The measuring device according to claim 1, having regard to claims 7 and 9,
wherein it exhibits both the first and the second first gas constituent observation wall component and a second gas constituent observation wall component with the second gas constituent foil layer, where at the housing the first and the second first gas constituent observation wall component are each arranged at a different side of the second gas constituent observation wall component.

13. The measuring device according to claim 1,
wherein at least at one longitudinal end of the housing, preferentially at two opposite longitudinal ends, a connector formation is arranged at each which is configured for connecting a hose and/or pipe line.

14. An bservation wall component for a measuring device, said measuring device comprising a housing surrounding the measuring chamber, of which at least one housing wall section is configured as an observation section for the acquisition of electromagnetic radiation emitted from the observation section in a direction away from the measuring chamber, where the observation section comprises at least one foil layer and where the housing is configured as a synthetic material injection molded housing, wherein the observation section exhibits at least one observation wall component, comprising an observation injection-molded body injected onto the at least one foil layer, where the housing comprises the observation wall component and a frame injection-molded body injected onto the observation wall component
wherein the observation wall component exhibits at least one foil layer with observation injection-molded bodies injected onto it, where the at least one foil layer exhibits a photoluminescent layer with at least one luminophore accommodated in it and/or where the observation injection-molded body exhibits an observation cutout, within which the at least one foil layer is accessible, where one lateral face of the observation cutout tapers in a direction towards at least one foil layer and thus is configured as a centering surface for tool engagement.

15. A method for fabricating an observation section of a measuring device for measuring a gas constituent of a gas present in a measuring chamber of the measuring device, where the measuring chamber is surrounded at least in part by the observation section, the method comprising the following steps:
inlaying of a foil body with at least oner foil layer in an injection-molding cavity,
injection of an observation injection-molded body onto the foil body and thereby forming an observation wall component, and
injection of a second injection-molded synthetic body onto the observation wall component and thereby fabricating the observation section.

16. The measuring device according to claim 5, wherein the at least one foil layer comprises a biaxially oriented polyolefin foil.

17. The measuring device according to claim 5,
wherein the at least one foil layer comprises a biaxially oriented polypropylene foil.

18. The measuring device according to claim 1,
wherein the at least one foil layer comprises as a temperature measurement foil layer a metal foil coated with black material.

19. The measuring device according to claim 1,
wherein the at least one foil layer comprises as a temperature measurement foil layer a aluminum foil coated with black material.

20. The measuring device according to claim 1,
wherein at at two opposite longitudinal ends, a connector formation is arranged at each which is configured for connecting a hose and/or pipe line.
